(19) United States
(12) Patent Application Publication
(10) Pub. No.: US 2021/0220432 A1
(43) Pub. Date: Jul. 22, 2021

(54) Application of Polypeptide in Preparation of Composition for Preventing and Treating Human Papillomavirus Infection

(71) Applicant: GENLOCI BIOTECHNOLOGIES INC., Nanjing (CN)

(72) Inventors: Jianqun LING, Nanjing (CN); Yixing WANG, Nanjing (CN); Qihui TANG, Nanjing (CN)

(73) Assignee: GENLOCI BIOTECHNOLOGIES INC., Nanjing (CN)

(21) Appl. No.: 16/768,962

(22) PCT Filed: Nov. 1, 2019
(86) PCT No.: PCT/CN2019/114968

§ 371 Date: Jun. 2, 2020

(30) Foreign Application Priority Data
Dec. 29, 2018 (CN) 201811652712.6

(51) Int. Cl. A61K 38/10; A61P 31/20

(52) U.S. Cl. CPC A61K 38/10; A61K 9/02; A61P 31/20

(57) ABSTRACT

An application of a polypeptide in preparing a composition for preventing and treating human papillomavirus infection, and a polypeptide composition for preventing and treating human papillomavirus infection are provided. The polypeptide composition is prepared by taking an effective dose of the polypeptide as an active ingredient, and adding a pharmaceutically acceptable adjuvant or auxiliary ingredient. The polypeptide of the present invention can be used for the treatment and prevention of human papillomavirus infection, having significant effect, no inhibitory effect on the normal flora of the female genital tract, and showing good safety.

### CROSS REFERENCE TO THE RELATED APPLICATIONS

This application is the national phase entry of International Application No. PCT/CN2019/114968, filed on Nov. 1, 2019, which is based upon and claims priority to Chinese Patent Application No. 201811652712.6, filed on Dec. 29, 2018, the entire contents of which are incorporated herein by reference.

### TECHNICAL FIELD

The present invention relates to a novel antiviral polypeptide in the technical field of medicine, and more specifically to an application of a polypeptide for preventing and controlling human papillomavirus infection.

### BACKGROUND

Human papillomavirus (HPV) belongs to the papillomaviridae family, which is a type of epitheliotropic, non-enveloped, double-stranded DNA virus that can cause squamous epithelium proliferation in human skin mucosa, manifested as common warts, genital warts (condyloma acuminatum) and other symptoms. With the rapid increase in the incidence of condyloma acuminatum in sexually transmitted diseases and the increase of cervical cancer, anal cancer and other cancers, HPV infection has attracted more and more attention. There are many subtypes of HPV. HPV isolated from human skin and mucosal tissues is divided into more than 100 subtypes, and can be classified into “low-risk” and “high-risk” types according to their carcinogenicity. The low-risk types (such as types 6 and 11) are mainly associated with genital warts and low-grade cervical epithelial necrosis. The high-risk types are represented by types 16 and 18, and longstanding infection with them is the major cause of tissue malignant tumors, especially cervical cancer.

At present, there are relatively few medications available in the world that are effective in preventing and controlling HPV infection. Medications for the treatment of cervical HPV infection commonly include interferon such as Xinfuning (recombinant human interferon α2b vaginal effervescent capsule), which can inhibit the replication and transcription of viral nucleic acid by inducing the production of antiviral protein with enzymatic activity in target cells, but its curative effect and toxic side effects are still considered controversial. The prophylactic HPV vaccine helps women prevent infection with the most common HPV subtypes associated with cervical cancer, but not with all types of virus strains, and the vaccine is expensive. In addition, the prophylactic HPV vaccine has no therapeutic effect on women who have been infected with related HPV subtypes. Therefore, there is an urgent need to develop a novel pharmaceutical preparation to prevent and control HPV.

Antimicrobial polypeptides are a kind of basic polypeptide, generally composed of about 12 to 50 amino acids, with broad-spectrum antibacterial activity. In addition to antibacterial effects, different types of antimicrobial polypeptides also have antiviral, antiprotozoal, anticancer, inflammation regulation, wound healing promotion, immune regulation and other effects. HPV is a non-enveloped virus, and therefore, initially, antimicrobial polypeptides were considered to have no inhibitory effect on HPV, because antimicrobial polypeptides generally play a role in resisting and inhibiting microorganisms through cell membranes. However, subsequent studies have shown that some antimicrobial polypeptides are effective against non-enveloped viruses, and their antiviral mechanism against non-enveloped viruses may be different from that of enveloped viruses. But even so, the effects of antibacterial polypeptides with different homologous sequences on HPV may be different. HD5 and HD6 belong to human α-defensin, and are secreted by human Paneth cells that are specialized secretory cells located within the folds of the small intestinal lining. One study found that HD5 can block HPV virion escape from endocytic vesicles, thereby inhibiting HPV activity, while HD6 remained inactive against HPV at the highest cytotoxic dose (Buck C B, Day P M, et al., Human alpha-defensins block papillomavirus infection. Proc Natl Acad Sci USA. 2006; 103(5):1516-21).

The polypeptide of the present invention was discovered by the applicant during the research. The polypeptide is an antimicrobial polypeptide, which has not been reported to have the activity of inhibiting HPV by the prior art. The polypeptide has a significant inhibitory effect on HPV infection, which has been proved by the applicant via experimental detection.

### SUMMARY

The applicant found that the polypeptide of the present invention has a significant inhibitory effect on human papillomavirus. In view of the drawbacks of the prior art, the primary objective of the present invention is to provide an application of a polypeptide in preparation of a composition for preventing and treating HPV infection. The second objective of the present invention is to provide a polypeptide composition for preventing and treating HPV infection. The polypeptide composition is prepared by taking an effective dose of a polypeptide as an active ingredient, and adding a pharmaceutically acceptable adjuvant or auxiliary ingredient. The third objective of the present invention is to provide a method for preventing and treating HPV infection.

The amino acid sequence of the polypeptide of the present invention is SEQ ID: NO.1.

The contents mentioned in the present invention are mass percentages unless otherwise specified.

In a first embodiment, the present invention provides an application of a polypeptide in preparation of a composition for preventing and treating HPV infection, wherein a concentration of the polypeptide is 0.01%-1%.

In a second embodiment, the present invention provides an application of a polypeptide in preparation of a composition for preventing and treating HPV infection, wherein a concentration of the polypeptide is 0.02%-0.2%.

In a third embodiment, the present invention provides a polypeptide composition for preventing and treating HPV infection, wherein the polypeptide composition is a composition prepared by taking an effective dose of a polypeptide as an active ingredient, and adding a pharmaceutically acceptable adjuvant or auxiliary ingredient.

In a fourth embodiment, the polypeptide composition of the present invention can be administered in any suitable form, for example, topical administration. The polypeptide composition may be any one of a liquid composition, an emulsion (an oil-in-water, a water-in-oil, an aerosol or a foaming agent), an ointment, a paste, a lotion, a powder, a liniment, a gel, a hydrogel, a hydrocolloid and a cream, and may be prepared to contain liposomes, micelles and/or microspheres.

In a fifth embodiment, a polypeptide composition suitable for vaginal administration may be a vaginal suppository, a soft capsule, an effervescent tablet, a gel, an ointment, a vaginal tablet, a film agent, or a foaming agent.

In a sixth embodiment, the present invention provides a polypeptide composition for preventing and treating HPV infection, wherein the polypeptide has a concentration of 0.01%-1%.

In a seventh embodiment, the present invention provides a polypeptide composition for preventing and treating HPV infection, wherein the polypeptide has a concentration of 0.02%-0.2%.

In an eighth embodiment, the present invention relates to a method for preventing and treating HPV infection. The method can be used to prevent and treat HPV-induced common warts, genital warts (condyloma acuminatum), flat warts, plantar warts, vulvar cancer, penile cancer, prostate cancer, oral cancer and other diseases. The polypeptide of the present invention has a better inhibitory effect on HPV, and the administration of the polypeptide composition of the present invention to the HPV infection site can prevent and treat HPV infection.

In a ninth embodiment, the polypeptide composition of the present invention for preventing and treating HPV infection can be made into a cleansing fluid for men and women, respectively, to clear the HPV infection in men and women, and to cut off the transmission of HPV through sexual contact or other ways.

The present invention provides a new use of a polypeptide for preparing a composition for the prevention and treatment of HPV infection. In addition to having an HPV inhibitory effect, the polypeptide composition may also have effects of regulating the immune system and promoting the healing of ulceration. Compared with the prior art, the polypeptide composition has a better treatment effect on HPV infection, and has no inhibitory effect on probiotics of female genital tract. The polypeptide of the present invention has high safety, and the final degradation product is an amino acid without any adverse reaction.

### BRIEF DESCRIPTION OF THE DRAWINGS

FIG. 1 is a sensorgram showing protein-protein interaction between a polypeptide and HPV16-L1; in the figure, 0 n, 3.125 n, 6.25 n, 12.5 n, 25.0 n, 50.0 n and 100 n represent the concentration of the polypeptide is 0 mg/L, 3.125 mg/L, 6.25 mg/L, 12.5 mg/L, 25.0 mg/L, 50.0 mg/L and 100 mg/L, respectively;

FIG. 2 shows results of a bacteriostatic test of a polypeptide against E. coli; and

FIG. 3 shows results of a bacteriostatic test of a polypeptide against Lactobacillus.

### DETAILED DESCRIPTION OF THE EMBODIMENTS

The present invention provides an application of a polypeptide in preparation of a composition for preventing and treating HPV infection. Unless otherwise specified, the polypeptides all have the amino acid sequence of SEQ ID: NO.1.

Unless otherwise specified, the technical means used in the embodiments are conventional means well known to those skilled in the art, and the raw materials added in the embodiments are commercially available.

The polypeptide of the present invention is an antimicrobial polypeptide, and the existing data only disclose that the polypeptide has antibacterial effect. During the study, the applicant unexpectedly discovered that the polypeptide of the present invention has an inhibitory effect on HPV. The present invention provides a polypeptide composition for preventing and treating HPV infection, and the polypeptide composition is a composition prepared by taking an effective dose of a polypeptide as an active ingredient, and adding a pharmaceutically acceptable adjuvant or auxiliary ingredient.

The “effective dose” mentioned in the present invention refers to the amount of an active ingredient contained in the polypeptide composition of the present invention that exhibits a preventive or therapeutic effect on a disease. The effective dose can be changed according to the patient's age, gender, administration site, administration frequency, administration time, dosage form, and type of adjuvant. Those skilled in the art will understand that the “effective dose” may vary depending on the mode of administration, the use of a carrier, the possible combination with other therapeutic agents, and other situations. The effective dose can be easily determined by one of ordinary skill in the art by conducting a limited number of dose response experiments, for example, applying a series of concentrations of a given formulation to a specific site on the body.

“Pharmaceutically acceptable adjuvant or auxiliary ingredient” will not destroy the anti-HPV activity of the polypeptide of the present invention. In addition, the amount of the pharmaceutically acceptable adjuvant or auxiliary ingredient that can play the role of pharmaceutical auxiliary materials or auxiliary functions is non-toxic and harmless to the human body. The polypeptide composition may contain any suitable carrier, diluent or excipient, including all commonly used solvents, dispersion media, fillers, solid carriers, antifungal and antibacterial agents, viscosity enhancers, film-forming agents, dermal penetrants, surfactants, isotonic agents, absorbents, and others.

In the present invention, a polypeptide composition suitable for vaginal administration may be a vaginal suppository, a lotion, a soft capsule, an effervescent tablet, a gel, an ointment, a vaginal tablet, a film agent, or a foaming agent. The knowledge of preparing different compositions by adding effective chemical or biological ingredients with an adjuvant, a carrier or an auxiliary ingredient is an available technology well known to those skilled in the art, for instance reference can be made to “Pharmaceutics”, “Pharmaceutical Structure and Preparation”, “Pharmaceutical Preparation Chemistry”, “Biopharmaceutics and Pharmacokinetics”, and others. Therefore, in the embodiments of the present invention, only the formulations and production processes of products in partial dosage forms are listed.

The polypeptide of the present invention can be obtained by chemical synthesis, or by expression, isolation and purification using genetic engineering techniques (for specific methods, see Sambrook et al., Molecular Cloning: A Laboratory Manual, Cold Spring Harbor Laboratory Press, Cold Spring Harbor, N.Y., 1989).

The embodiments in this section further illustrate the contents of the present invention, but should not be construed as a limitation of the present invention. Without departing from the spirit and essence of the present invention, any modification or replacement made to the methods, steps, or conditions of the present invention shall fall within the scope of the present invention.

The present invention discloses for the first time the application of the polypeptide as a pharmaceutical active ingredient in preparing the composition for treating and preventing HPV infection. Therefore, using the polypeptide of the present invention alone or in combination with other substances as a pharmaceutical active ingredient together with an adjuvant to prepare a medicament, as long as the medicament is used for the treatment and prevention of HPV infection, all fall within the protective scope of the present invention.

### Embodiment 1 Preparation of polypeptide Vaginal Gel

(1) The formula is as follows:
polypeptide, 0.04%;
hydroxypropyl methylcellulose, 2%;
ethylhexylglycerol, 0.3%; and
purified water, balance.

(2) Process Flow

The hydroxypropyl methylcellulose is swollen, and then heated and redissolved to obtain a solution-like substrate. When the solution-like substrate is at 80 degrees, the polypeptide and ethylhexylglycerol are added in sequence, followed by thoroughly stirring and mixing, and adjusting pH to 5.5 to prepare the polypeptide vaginal gel.

### Embodiment 2 Preparation of polypeptide Cleansing Solution

(1) The formula is as follows:
polypeptide, 0.2%;
glycerin, 0.5%;
ethylparaben, 0.02%;
ethanol, 0.32%;
arginine, 0.6%;
lysine, 5%; and
purified water, balance.

(2) Process Flow

The ethylparaben is dissolved in ethanol for subsequent use, and the glycerin is premixed and stirred for subsequent use. The polypeptide, arginine and lysine are mixed in purified water, then added into the glycerin solution in the previous step, and then the ethylparaben solution is added, followed by stirring uniformly, and adjusting pH to 6.2 to prepare the polypeptide cleansing solution.

### Embodiment 3 Protein-Protein Interaction Relationship Between the polypeptide and HPV Antigen

The HPV capsid is composed of two structural proteins, L1 and L2. The L1 alone or L1 and L2 after co-expression can be assembled into virus-like particles. The L1 protein is highly conserved among all types of HPV, and is involved in the packaging of intact HPV virus particles and the binding of HPV to host cell receptors.

Biomolecular interaction analysis (BIA) is a biosensing analysis technology based on a physical optical phenomenon known as surface plasmon resonance (SPR). BIA can provide real-time observation of the interaction between biomolecules, through which, the binding specificity of two molecules can be observed, how strong the binding of the two molecules is can be known, and how many cooperators and participants there are in the binding process of biomolecules can be known.

In this experiment, BIA technology is used to observe whether there is an interaction between the polypeptide of the present invention and the HPV L1 protein, so as to explore the mechanism of inhibiting HPV virus by the polypeptide.

1. Experimental Reagents:
Ac 4.0: 10 mM sodium acetate-acetic acid buffer, PH 4.0.

2. Coupling Reagent (Amino Coupling):
EDC (N-ethyl-N′-(3-dimethylaminopropyl)carbodiimide hydrochloride, Cas: 25952-53-8): 0.4 M, 750 mg of the EDC is dissolved with 10 mL of ddH₂O, and is formulated immediately before use;
NHS (N-Hydroxysuccinimide, Cas: 6066-82-6): 0.1M, 115 mg of the NHS is dissolved with 10 mL of ddH₂O, and is formulated immediately before use;
Ethanolamine hydrochloride: 975.4 mg of the Ethanolamine hydrochloride is dissolved in 0.1 M sodium borate (PH 8.5) to a final volume of 10 mL;
HBS-EP buffer: 0.01 M Hepes (PH7.4), 150 mM NaCl, 3 mM EDTA, 0.05% (v/v) P20; and
Renew Buffer: 50 mM NaOH.

3. Main Materials:
CMS chip coupled with recombinantly expressed HPV-16 L1 protein; and
the polypeptide.

4. Preparation of the chip: HPV-16 L1 protein (Cat. No. ab119880) purchased from the Abcam company is used, and the protein in the form of lyophilized powder is dissolved in a small amount of running buffer (HBS-EP). The coupling is performed using the EDC/NHS method. The binding amount of HPV-16 L1 protein is about 5300 Ru.

5. Experimental Procedure

(1) The instrument preparation and preheating are completed according to the Biacore 3000 manual; and the chip preparation and solution equilibration are completed with reference to the instructions of the CMS chip.

(2) The purchased HPV-16 L1 protein lyophilized powder is diluted with purified water to obtain a 1 mg/mL protein solution; and the protein solution was diluted with Ac 4.0 solution to obtain a working solution with a final concentration of 0.05 mg/mL; HPV-16 L1 protein is used as a target protein for coupling.

(3) The Surface Preparation/Immobilization command in the Biacore 3000 operating software is used, and the coupling operation of the target protein on the CMS chip is performed according to the prompt. The coupling method is NHS/EDC-mediated amino coupling reaction. In this reaction, the carboxyl group on the CMS chip is activated by using the NHS/EDC reagent to enable the carboxyl group to react with amino acids or the amino terminus of the protein to form peptide bonds at normal temperature.

(4) Through steps 1-3, a blank channel is similarly prepared as a control for the experimental channel. The blank channel is not suitable for coupling with amino acid or protein. The original carboxyl group on the channel is finally bonded to ethanolamine and converted into a hydroxyl group.

(5) The coupling effect is automatically calculated by the software. In the experiment, the experimental channel is finally coupled with about 5300 RU of HPV-16 L1 Protein, where the unit RU is the unit of coupling quantity defined by the system.

(6) The chip and the equipment are fully equilibrated using the running buffer, i.e., HBS-EP buffer.

(7) The peptides to be tested are formulated with HBS-EP buffer into a solution with a final concentration of 0 mg/L, 3.125 mg/L, 6.25 mg/L, 12.5 mg/L, 25.0 mg/L, 50.0 mg/L and 100 mg/L, respectively, for subsequent experiments. In order to facilitate the experiment operation, “mg/L” is replaced with “n” for the standard.

(8) The program is designed according to the steps of equilibration, testing, equilibration, and regeneration, and the polypeptide solution of each concentration was detected. The detection procedure is as follows: equilibration for 120 s, injection for 180 s, equilibration for 540 s, regeneration with Renew Buffer for 60 s, and equilibration for 60 s. During the detection, the sample passes through the blank channel and the experimental channel in turn, and the data to be detected is the resultant difference of “experimental channel response value minus blank channel response value”.

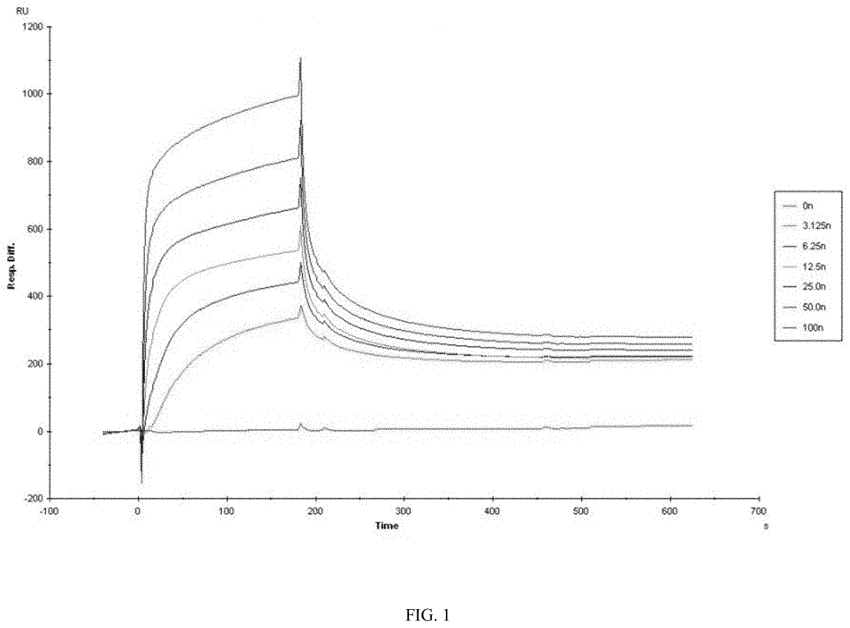

(9) For the obtained data, the supporting data analysis software BIAEVAL of Biacore 3000 is used for mapping and data analysis, and the test results are drawn into a sensorgram showing the protein-protein interaction. The sensing and calculation results are shown in FIG. 1 and Table 1.

TABLE 1
Results of data analysis

| Item | Value | Annotation |
|---|---|---|
| ka (1/Ms) | 2.35E+06 | Binding constant |
| kd (1/s) | 1.84E−03 | Dissociation constant |
| Rmax (RU) | 414 | Maximum binding capacity |
| KD (M) | 7.82E−10 | Affinity constant |
| *Chi2 | 11.1 | Chi-square test after data |
| | | normalization |

6. Results

The affinity constant between the tested polypeptide and the target protein (HPV-16 L1 protein) is obtained. The affinity constant of the polypeptide binding to the HPV16 L1 protein is 7.82×10⁻¹⁰ M.

7. Conclusion:

With reference to the existing Biacore method, the affinity constants determined by kinetic analysis of anti-HPV antibodies and HPV L1 protein are analyzed. Affinity constants of antibodies and antigens on the order of 10⁻¹⁰ M are considered as high affinity. The affinity constant of the polypeptide determined in this experiment and HPV-16 L1 protein is also up to the order of 10¹⁰ M, indicating that the polypeptide determined has a strong affinity to the HPVL1 protein. Therefore, the mechanism by which the polypeptide inhibits the activity of HPV is inferred as follows: the binding of the polypeptide to the L1 coat protein of HPV, on the one hand, affects the assembly of intact HPV virus particles, and on the other hand, blocks the binding site of HPV to the host cell receptor, so that HPV cannot bind to the host cell, thereby inhibiting the activity of HPV.

### Embodiment 4 Inhibition of HPV6 and 11 by the polypeptide

Experimental Materials: Source of HPV6/11 virus: HPV6 and 11 viruses are taken from the prostate fluid specimens of untreated patients with condyloma acuminatum, and the specimens are transferred into 1.5 mL centrifuge tube, evenly mixed by shaking, and centrifuged at 12000 rpm for 10 min, then the supernatants are discarded, and the precipitations are taken for subsequent use.

Test substance: the polypeptide is dissolved in physiological saline and prepared into polypeptide solutions having concentrations of 0.01%, 0.5% and 1%.

Main instrument: ABI 7500 nucleic acid testing instrument.

Kit: low-risk type (HPV6/11), produced by the DaAn Gene Co., Ltd. of Sun Yat-sen University.

Experimental method: 10 of the test substance is added to the prostate fluid precipitation, and 10 μL of normal saline is added to a control tube, both negative and positive control tubes are prepared for each experiment, and followed by incubating at 37° C. for 24 hours. 50 of DNA extraction solution is added to the sample and mixed well, then lysed at a constant temperature of 100° C. for 10 min, and then centrifuged at 12000 rpm for 5 min for subsequent use. 5 of nucleic acid extraction supernatant is added to the Polymerase chain reaction (PCR) system, centrifuged at 8000 rpm for several seconds, and then placed in the sample cell of the instrument for PCR experiment. The reaction cycle conditions are as follows: 93° C. for 2 min, followed by 10 cycles at 93° C. for 45 s and 55° C. for 60 s, 30 cycles at 93° C. for 30 s and 55° C. for 45 s. The analysis results are shown in Table 2.

TABLE 2
Results of quantitative detection of
HPV6/11 in prostate fluid by FQ-PCR

| Test substance | Concentration | Value |
|---|---|---|
| Blank control | — | 6.74 × 10⁷ |
| The polypeptide | 0.01% | 3.86 × 10⁴ |
| | 0.5% | <2.5 × 10³ |
| | 0.1% | <2.5 × 10³ |

As can be seen from the results in Table 2, 24 hours after adding 0.01% of the polypeptide to the prostate fluid of patient with condyloma acuminatum, HPV6/11 has decreased significantly, and 0.1% of the polypeptide reduces the amount of HPV virus to below the detection limit, that is negative; however, the blank control tube has a high concentration of HPV virus. The results show that the polypeptide can rapidly and effectively inhibit the HPV6/11 virus causing condyloma acuminatum.

### Embodiment 5 Therapeutic Effect of the Composition Containing the polypeptide of the Present Invention on Various HPV Subtype Infections

Seventy-six patients with high-risk cervical infection are selected. The high-risk HPV subtypes are HPV type 16, 18 or 52. All patients are randomly divided into a control group and an observation group, with 38 patients in each group.

Inclusion criteria: All patients are tested (1) positive by thin-layer liquid-based cytology (known as Thinprep Cytologic Test, TCT); (2) positive by HPV test; and (3) not to have serious diseases in heart, liver, kidney and other important organs.

Exclusion criteria: (1) patients with concurrent malignant tumors and systemic immune system diseases; (2) patients with contraindications to interferon; (3) patients having a history of reproductive system surgery; (4) patients with cognitive impairment; and (5) patients during pregnancy or lactation.

Method: in the control group, after cleaning the vulva before going to bed, one tablet of Xinfuning is placed on the posterior fornix or vaginal cuff, once a day, 10 days as a course of treatment, a total of 6 courses. In the observation group, after cleaning the vulva before going to bed, one tube (5 mL) of the polypeptide gel composition described in embodiment 1 of the present invention is pushed into the posterior fornix or vaginal cuff, one tube per day, stand up after sitting or lying for 5 minutes, stop using during menstruation, and using for 60 days.

Observation indicators: after treatment, the high-risk HPV test shows that the original subtype turns negative, which is effective; and the high-risk HPV test shows that the original subtype does not turn negative, which is invalid.

The results of the control group and the observation group after treatment are shown in Table 3.

TABLE 3
Comparison of negative conversion ratio
between two groups of treatment results

| | Number of | | | Negative |
|---|---|---|---|---|
| | patients | Effective | Invalid | conversion ratio |
| Group | (person) | (person) | (person) | (%) |
| Control group | 38 | 26 | 12 | 68.4 |
| Observation group | 38 | 34 | 4 | 89.5 |

The results in Table 3 show that the negative conversion ratio of the observation group using the gel composition containing the polypeptide of the present invention is as high as 89.5%, the negative conversion ratio of the control group is 68.4%, and the difference is statistically significant (P<0.05). The above clinical experimental studies fully show that the composition containing the polypeptide of the present invention can effectively treat HPV infection, and can further prevent and treat cervical cancer.

### Embodiment 6 the Effect of the polypeptide on Vaginal Probiotics

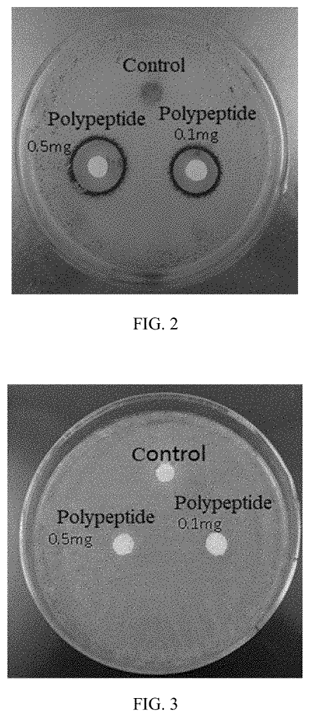

The polypeptide of the present invention has an antibacterial effect on a variety of bacteria, but the polypeptide has no inhibitory effect on the normal flora of the female genital tract, such as Lactobacillus. In FIG. 2, the polypeptides at concentrations of 0.1 mg and 0.5 mg have antibacterial effect on Escherichia coli, with the zone of inhibition having a diameter of greater than 7 mm, respectively. In FIG. 3, the polypeptides at concentrations of 0.1 mg and 0.5 mg have no antibacterial effect on Lactobacillus, do not form zone of inhibition. It can be seen that, while inhibiting HPV, the polypeptide can effectively maintain the balance of probiotic microenvironment in the female genital tract without affecting the growth of beneficial vaginal flora.

The above embodiments are preferred embodiments of the present invention, but the implementing methods of the present invention are not limited by the above embodiments. Any other changes, modifications, substitutions, combinations, and simplifications made without departing from the spirit and principle of the present invention shall be equivalent replacement methods, and shall be included in the protective scope of the present invention.

What is claimed is:

1. A method of preparing a composition for preventing and treating human papillomavirus infection, comprising: using a polypeptide, wherein, an amino acid sequence of the polypeptide is SEQ ID NO:1.

2. The method according to claim 1, wherein, a concentration of the polypeptide is 0.01%-1%.

3. The method according to claim 2, wherein, the concentration of the polypeptide is 0.02%-0.2%.

4. The method according to claim 1, wherein, the composition is an external dosage form, the composition is formed by compounding the polypeptide with a water-soluble substrate or a fat-soluble substrate, and the external dosage form comprises at least one of a gel agent, a liquid, a suppository, an effervescent suppository, an effervescent capsule, a soft capsule, an effervescent tablet and a sponge suppository.

5. A polypeptide composition for preventing and treating human papillomavirus infection, wherein, the polypeptide composition is prepared by adding an effective dose of the polypeptide of claim 1 as an active ingredient with a pharmaceutically acceptable adjuvant or auxiliary ingredient.

6. The polypeptide composition according to claim 5, wherein, a concentration of the polypeptide is 0.01%-1%.

7. The polypeptide composition according to claim 6, wherein, the concentration of the polypeptide is 0.02%-0.2%.

8. The polypeptide composition according to claim 5, wherein, the polypeptide composition is an external dosage form, the polypeptide composition is formed by compounding the polypeptide with a water-soluble substrate or a fat-soluble substrate, and the external dosage form comprises at least one of a gel agent, a liquid, a suppository, an effervescent suppository, an effervescent capsule, a soft capsule, an effervescent tablet and a sponge suppository.

9. The method according to claim 2, wherein, the composition is an external dosage form, the composition is formed by compounding the polypeptide with a water-soluble substrate or a fat-soluble substrate, and the external dosage form comprises at least one of a gel agent, a liquid, a suppository, an effervescent suppository, an effervescent capsule, a soft capsule, an effervescent tablet and a sponge suppository.

10. The method according to claim 3, wherein, the composition is an external dosage form, the composition is formed by compounding the polypeptide with a water-soluble substrate or a fat-soluble substrate, and the external dosage form comprises at least one of a gel agent, a liquid, a suppository, an effervescent suppository, an effervescent capsule, a soft capsule, an effervescent tablet and a sponge suppository.

11. The polypeptide composition according to claim 6, wherein, the polypeptide composition is an external dosage form, the polypeptide composition is formed by compounding the polypeptide with a water-soluble substrate or a fat-soluble substrate, and the external dosage form comprises at least one of a gel agent, a liquid, a suppository, an effervescent suppository, an effervescent capsule, a soft capsule, an effervescent tablet and a sponge suppository.

12. The polypeptide composition according to claim 7, wherein, the polypeptide composition is an external dosage form, the polypeptide composition is formed by compounding the polypeptide with a water-soluble substrate or a fat-soluble substrate, and the external dosage form comprises at least one of a gel agent, a liquid, a suppository, an effervescent suppository, an effervescent capsule, a soft capsule, an effervescent tablet and a sponge suppository.


SEQUENCE LISTING (1 sequence)

SEQ ID NO: 1 - protein, 19 aa, Artificial Sequence
  The sequence is a synthetized amino acid sequence
MGRFKRFRKKFKKLFKKLS